(19) United States
(12) Patent Application Publication
(10) Pub. No.: US 2007/0048433 A1
(43) Pub. Date: Mar. 1, 2007

(54) Coating of surgical devices

(76) Inventors: Martin David Hallett, Gravesend (GB); Marshall Whiteman, Ditton (GB); Ian James Stringer, Dartford (GB); Linda Green, Orpington (GB)

Correspondence Address: Robert C Klinger;Jackson Walker, Suite 600, 2435 North Central Expressway, Richardson, TX, 75080, US

(21) Appl. No.: 10/567,229

(22) PCT Filed: Aug. 5, 2004
(86) PCT No.: PCT/GB04/03405

§ 371 Date: Sep. 18, 2006

(30) Foreign Application Priority Data
Aug. 5, 2003 (GB) 0318353.0

(51) Int. Cl. A61L 33/00; B05D 3/00; B05D 1/04; H05C 1/00

(52) U.S. Cl. 427/2.1; 427/458

(57) ABSTRACT

The invention provides a method for the coating of a surgical device, wherein the coating is carried out by electrostatic Powder deposition. The device may be a device used in a surgical or diagnostic procedure, including interventional devices as well as implantable devices. Because the coating is applied electrostatically, it is attracted to all parts of the device, not just those parts that are in the ‘line of sight’ of the spray, as is the case with conventional liquid spray coating. The process allows uniform and reproducible amounts to be deposited and thus drug-eluting coatings can be accurately applied to stents and other surgical devices, resulting in good control over drug release. Furthermore, drug-eluting coats can be applied in a single step, although multiple layers can easily be applied if desired to create a specific drug release profile.

This invention relates to the coating of surgical devices, more especially, but not exclusively, for coating stents and heart valves. Other devices include pacemakers, catheters, orthopaedic and dental implants, artificial hips and other joints, artificial organs, neurostimulators, cardiovert defibrillators and tubing used in dialysis and in heart lung machines.

Such devices are inserted in the body to treat a variety of medical conditions, but are “foreign objects” to the body, and can lead to immune and other responses and reactions, so that drug treatments to suppress the immune and protective responses of the body have been proposed. Such treatments, however, have serious risks and in recent years efforts have been made to use biocompatible materials that do not provoke an abnormal inflammatory response and do not lead to allergic or immunologic reaction. Accordingly, the use of devices made from or coated with biocompatible materials (biomaterials) is steadily increasing in modern healthcare, and alternative drug-delivery systems that bring medication to targeted areas in the body are also widely sold.

Stents are small mechanical devices that can be implanted in body structures such as vessels, tracts or ducts, for example in blood vessels, the urinary tract and in the bile duct, to treat these body structures when they have weakened. With blood vessels, stents are typically implanted therein to treat narrowings or occlusions caused by disease, to reinforce the vessel from collapse or to prevent the vessel from abnormally dilating, as, for example, with an aneurysm.

Typically, a stent comprising a mesh or perforated tube is inserted directly to the site of closure or narrowing, and is mechanically expanded by, for instance, a balloon to reopen the vessel at the site of closure.

There has been explosive growth in the use of coronary stents in interventional cardiology, with stents being used in as many as 80% of cases in some major centres. Recently, there has been considerable interest in stent coating for local delivery of drugs and prevention of restenosis, and a biocompatible coating layer is often used as a drug carrying layer. The materials used may either be synthetic (e.g. polyurethane, poly-L lactic acid, Dacron, polyester, polytetrafluoroethylene (PTFE), poly(ethyl acrylate)/poly(methyl methacrylate), polyvinyl chloride, silicone, collagen or iridium oxide) or naturally occurring substances (e.g. heparin, phosporylcholine).

Stents are generally of metal construction and come in a variety of designs. These include self-expanding stents, balloon expandable coil stents, balloon expandable tubular stents and balloon expandable hybrid stents. The metal is usually stainless steel, but cobalt alloy, Nitinol® and tantalum, for example, are also used. Other devices such as heart valves are also made of metal, and there is generally a need for a pre-treatment step to ensure the adherence of the coating to the metal substrate, more especially in the case of a polymeric coating.

Moreover, the design of stents in particular is generally complex, making the devices inherently difficult to coat in a uniform and reproducible way by conventional means. Coatings are usually multilayers, and drug-containing layers are usually applied by a technically crude spraying process. For example, coating may be carried out by a process involving immersion coating (dip coating) to produce a primer layer, followed by aerosol spraying of the drug-loaded material onto the primer coating. Heat shrinking or vapour deposition may alternatively be used to apply the coating material onto the stent.

Such methods lead to variability of the coatings applied, and the variability is compounded when multilayers are applied. In such circumstances, if the coating is variable, drug release will be poorly controlled; optimal drug delivery requires uniform, reproducibly coated stents. Similar problems of non-uniform coating of substrates, especially substrates of complex shape, are found in the production of other surgical devices, for example heart valves.

We have found that pharmaceutically acceptable coatings can be applied satisfactorily by electrostatic means to metal and other substrates of different shapes, allowing for the possibility of formation of a uniform and reproducible coating with and without a content of active material, and avoiding the need for a primer coating.

Accordingly, the present invention provides a method for the coating of a surgical device, wherein the coating is carried out by electrostatic powder deposition.

The method of the invention has a number of advantages. Because the coating is applied electrostatically, it is attracted to all parts of the device, not just those parts that are in the ‘line of sight’ of the spray, as is the case with conventional liquid spray coating. Moreover, the process is controllable and allows uniform and reproducible amounts to be deposited. Thus, drug-eluting coatings can be more accurately and consistently applied to stents and other surgical devices than by other techniques, resulting in much better control over drug release. Furthermore, drug-eluting coats can be applied in a single step, so there is no need for multiple coating layers, although multiple layers can easily be applied if desired to create a specific drug release profile.

The device may be a medical device used in a surgical or diagnostic procedure, including interventional devices as well as implantable devices, e.g. for intravascular placement, e.g. a device preferably for local delivery of an active material, and dental implants, neurostimulators and cardiovert-defibrillators should be mentioned.

Thus, the present invention also provides a medical device for implantation in the human or animal body or for a medical interventional procedure, which has been coated by electrostatic powder deposition.

The powder used for coating may include an active material which is delivered to the body after placement. The active material may be for administration to the human or animal body, for example for the prevention and/or treatment of a disease or other condition, as well as for example an active material administered in connection with a diagnostic or other investigation or interventional procedure.

The device may be a medical device made of metal, or may be an insulator material or semi-conductive material, e.g. plastic, ceramic, quartz, bioactive glasses, although such insulator and semi-conductive materials should generally be less than 1 mm in thickness.

The thickness of the coating on the device will generally be less than 100 microns, typically 30 microns or less.

Stents, for example, are manufactured at a first diameter and length for delivery and deployment, e.g. on a balloon catheter, and then expanded to a second, larger diameter upon placement at the requisite site, e.g. by expansion of the balloon portion of the balloon catheter. As many as 30 different stent designs are in use in the world. These can be classified according to structural characteristics of the stents, and include original slotted tube stents (e.g. Palmaz-Schatz), second generation tubular stents (e.g. Crown, MultiLink, NIR), self-expanding stents (e.g. Wallstent), coil stents (e.g. Crossflex, Gianturco-Roubin) and modular zigzag stents (e.g. AVE GFX). Stents may have diameters (unexpanded) for example ranging from approximately 1.255 mm to 4.75 mm, with lengths of approximately 5 mm to 60 mm.

The electrostatic application of powder material to a substrate is known. Methods have already been developed in the fields of electrophotography and electrography, and examples of suitable methods are described, for example, in Electrophotography and Development Physics, Revised Second Edition, by L. B. Schein, published by Laplacian Press, Morgan Hill Calif. The electrostatic application of powder material in the field of pharmaceuticals is also known, for example from WO 92/14451, WO 96/35413, WO 96/35516 and WO 98/20861. However, there has been no disclosure of such coating methods for stents or other devices for implantation in the body.

In the method of the present invention, preferably powder is deposited electrostatically on the shaped substrate, and then treated to form a continuous layer on the substrate, for example by IR and/or convection heating.

More especially the surgical device may comprise a metal substrate, for example stainless steel; a metal support provides an excellent substrate for electrostatic deposition because of its high conductivity. Stents, for example, are preferably made from thin walled metal tubing; suitable metals include stainless steel, Nitinol®, tantalum, platinum, and platinum/tungsten, which are biocompatible and radio-opaque. Other substrates include, for example, titanium alloys, and other possible devices include heart valves, pacemakers, catheters, orthopaedic implants, artificial joints, artificial organs, catheter sheaths and introducers, drug infusion catheters and guidewires.

Preferably the powder material is electrostatically charged and an electric field is present in the region of the device to cause the powder material to be deposited on the device. For example, the powder material may be electrostatically charged with a sign of one polarity, an electric potential of the same polarity may be maintained in the region of a source of the powder material and the device may be maintained at a lower, earth or opposite potential. For example, the powder material may be electrostatically charged positively, a positive potential may be maintained in the region of a source of the powder material and the device may be maintained at earth potential. The powder material may have a permanent or temporary net charge. Any suitable method may be used to charge the powder material. Advantageously, the electrostatic charge on the powder material is applied by triboelectric charging (as is common in conventional photocopying) or corona charging. The use of a charge-control agent encourages the particle to charge to a particular sign of charge and to a particular magnitude of charge.

The electric field is preferably provided by a bias voltage that is a steady DC voltage. Preferably, an alternating voltage, which is substantially higher than the DC voltage, is superimposed on the bias voltage. The alternating voltage preferably has a peak to peak value greater than, and more preferably more than twice, the peak value of the DC bias voltage. The DC bias voltage may be in the range of 100V to 2,000V and is preferably in the range of 200V to 1,200V. The alternating voltage may have a peak to peak value of the order of 5,000V and may have a frequency in the range of 1 to 15 kHz.

Achievement of good and even coating is facilitated if the spacing between the source of powder material and the device is relatively small, that is less than 10 mm, although spacings of up to 2 or 3 cm may also be possible. Preferably the spacing is in the range of 0.3 mm to 2 to 3 cm, e.g. up to 5 mm and more preferably between 0.5 mm to 5 mm.

The method may include the steps of:

applying a bias voltage to generate an electric field between a source of the powder material and the device;

applying the electrostatically charged powder material to the device, the powder material being driven onto the device by the interaction of the electric field with the charged powder material and the presence of the charged powder material on the device serving to build up an electric charge on the device and thereby reduce the electric field generated by the bias voltage between the source of powder material and the device, and

continuing the application of the electrostatically charged powder material to the device until the electric field between the source of powder material and the device is so small that the driving of the powder material by the electric field onto the device is substantially terminated.

Furthermore, charged material, already present on a partially coated device, will also alter the development field locally, which will tend to direct incoming material towards adjacent, less coated areas. This results in an even coating, even when the uncoated area is not in the line of sight of the powder source. Also using such a method promotes even coating of the device even when the spacing of some parts of the device from the source of powder material differs from the spacing of other parts. That is of particular advantage when the device is a complex shape. Furthermore the method promotes even coating regardless of the rate at which powder is deposited on the device and may be employed when there is relative movement between the device and the-source of powder material during deposition. In a case where the thickness of one layer of coating is not as great as the final thickness required, one or more other coating layers may be deposited and, if desired, the DC bias voltage increased for the deposition of the further layer(s), a layer being fused before the application of a further layer.

Selection of the physical arrangement to be employed for coating of the device is dependent upon the shape of the device to be coated. For example, it is possible to provide a plurality of separate sources of powder material to coat a single device and/or to provide sources of complex shapes and/or to provide electric fields of complex shapes. It is also possible to arrange for the source of powder material and/or the device to move during the application of the powder material. In the case where the device is of generally cylindrical shape, the source of powder material may be positioned at a radial spacing from the device and the device may be rotated relative to the source of powder material. A difference in spacing between the source and different parts of the device need not, however, result in uneven coating, especially if application of the powder material is continued until the electric field between the source of material and the device is substantially cancelled.

Further details of suitable methods and apparatus are described in WO 92/14451, WO 96/35516, WO 01/43727, WO 02/49771, WO 03/061841 and WO 04/24339, and in our copending applications PCT/GB 2004/002618, GB 0330171.0 and GB 0407312.8, the texts and drawings of which are incorporated herein by reference.

The present invention also provides an apparatus for coating a surgical device, the apparatus including a source of charged powder material and a voltage source for applying a bias voltage between the source of powder material and the device to generate an electric field therebetween such that powder material is applied to the device. Other optional features of the apparatus will be apparent from the description elsewhere of the method of the invention. The apparatus may be suitable for carrying out any of the methods described herein.

Powder coating materials suitable for electrostatic application and that are treatable on the substrate to form a film coating and processes for their use are disclosed, for example, in WO 96/35413, WO 98/20861, WO 98/20863 and WO 01/571144, the texts and drawings of which are incorporated herein by reference. Advantageously the powder material is prepared by melt extrusion of the components of the powder material or by other method producing particles comprising different component materials together in the particle.

Generally, the powder material includes a component which is fusible. Fusible coating materials include poly(vinylpyrrolidone), poly(bis(carboxylatophenoxy)-phosphazene), poly (acrylic acid), poly(methacrylic acid), poly(1-lysine), poly(ethylene glycol), poly(D-glucosamine), poly(1-glutamic acid), poly(diallyldimethylamine), poly(ethylenimine), hydroxy fullerene or long-sidechain fullerene, and combinations thereof. Poly-lactides, especially poly-L-lactides, may also be used, a specialised form of which (a high molecular weight poly-L-lactic acid) is biodegradable. PLLA (poly-L-lactide), PGA (poly-glycolide), PDLLA/PGA (poly-DL-lactide-co-glycolide), PLLA/PCL (poly-L-lactide-co-caprolactone), and PAA/cys(poly-acrylic acid-cysteine) should especially be mentioned. Examples of other biocompatible coatings include polyurethane, poly(butyl methacrylate-co-methyl methacrylate), polycaprolactone, polyethylene, PTFE, or TEFLON®, and phosphorylcholine.

Other suitable polymer binder components (also referred to as resins), include, e.g., acrylic polymers, e.g. methacrylate polymers, for example an ammonio-methacrylate copolymer; polyvinylpyrrolidone-vinyl acetate copolymers; polysaccharides, for example cellulose ethers and cellulose esters, e.g. hydroxypropyl cellulose, hydroxypropyl methylcellulose phthalate, hydroxypropyl methylcellulose and hydroxypropyl methylcellulose acetate succinate; phthalate derivatives of polymers. Others that should be mentioned include polyesters; polyurethanes; polyamides, for example nylons; polyureas; polysulphones; polyethers polystyrene; biodegradable polymers, for example polycaprolactones, polyanhydrides, polyglycolides, polyhydroxybutyrates and polyhydroxyvalerates; and also non-polymeric binders such as, for example, sugar alcohols, for example lactitol, sorbitol, xylitol, galactitol and maltitol; sugars, for example sucrose, dextrose, fructose, xylose and galactose; hydrophobic waxes and oils, for example vegetable oils and hydrogenated vegetable oils (saturated and unsaturated fatty acids), e.g. hydrogenated castor oil, carnauba wax, and bees wax; hydrophilic waxes; polyalkenes and polyalkene oxides; polyethylene glycol. Clearly there may be other suitable materials, and the above are given merely as examples. One or more fusible materials may be present. Preferred fusible materials generally function as a binder for other components in the powder. A polymer used may be one having release-rate controlling properties. Examples of such polymers include polymethacrylates, ethylcellulose, hydroxypropylmethyl-cellulose, methylcellulose, hydroxyethylcellulose, hydroxypropylcellulose, sodium carboxymethylcellulose, calcium carboxymethylcellulose, acrylic acid polymer, polyethylene glycol, polyethylene oxide, carrageenan, cellulose acetate, glyceryl monostearate, zein etc. Xylitol or other sugar alcohol may be added to the polymer binder, for example when the polymer binder is insoluble, to promote solubility. The fusible component may, if desired, comprise a polymer which is cured during the treatment, for example by heat curing or by irradiation with energy in the gamma, ultra violet or radio frequency bands. When different fusible materials are used, they are preferably compatible so that they can fuse together.

Advantageously, the powder material comprises a poly-lactide, polycaprdlactone, polyvinylpyrrolidone, poly(acrylic acid), poly(butyl methacrylate-co-methyl methacrylate), or polyurethane.

In general the powder material should contain at least 30%, usually at least 35%, advantageously at least 80%, by weight of material that is fusible, and, for example, fusible material may constitute up to 95%, e.g. up to 85%, by weight of the powder. Wax, if present, is usually present in an amount of no more than 6%, especially no more than 3%, by weight, and especially in an amount of at least 1% by weight, for example 1 to 6%, especially 1 to 3%, by weight of the powder material.

After application the powder coating may be converted into a coherent film by heating, preferably by infra-red radiation, but other forms of electromagnetic radiation or convection heating may be used. Usually the change in the coating upon heating will simply be a physical change. The powder material may be heated to a temperature above its softening point, and then allowed to cool to a temperature below its Tg to form a continuous solid coating. It may, for example, be heated to a temperature of 150 to 250° C., for example for 1 to 5 minutes, e.g. 3 to 4 minutes. Preferably, the powder material is fusible at a pressure of less than 100 lb/sq inch, preferably at atmospheric pressure, at a temperature of less than 250° C. Alternatively, for example, if the powder coating comprises a polymer which is curable, it may be treated by convection and/or IR heating and/or by irradiation with energy in the gamma, ultra-violet or radio frequency bands, to form a continuous cross-linked polymer coating.

The powder material may also contain, for example, one or more pharmacotherapeutical or diagnostic agents; for example a coating may contain an agent for the treatment or prevention of restenosis, an anticoagulant, an anti-thrombogenic agent, an anti-microbial agent, an anti-neoplastic agent, an antiplatelet agent, an immunosuppressant agent, an antimetabolite, an anti-proliferative agent, or an anti-inflammatory agent. The use of stents as a platform for the delivery of radiation to the vessel wall to combat in-stent restenosis should also be mentioned. Effective doses of radioactivity can be delivered to all levels of the vessel wall from stent-bound radioactive sources.

The powder material may advantageously also include a plasticiser to provide appropriate rheological properties. Examples of suitable plasticisers are ethyl citrate and polyethylene glycol. A plasticiser may be used with a resin in an amount, for example, of up to 50%, advantageously up to 30%, preferably up to 20%, by weight of the total of that resin and plasticiser, the amount depending inter alia on the particular plasticiser used. Plasticiser may be present, for example, in an amount of at least 2%, advantageously at least 5%, by weight based on the weight of the total powder material, and amounts of 2 to 30%, especially 5 to 20%, are preferred.

Preferably, the powder material includes a material having a charge-control function. That functionality may be incorporated into a polymer structure, as in the case of ammonio-methacrylate polymers mentioned above, and/or, for a faster rate of charging, may be provided by a separate charge-control additive. Examples of suitable charge-control agents are: metal salicylates, for example zinc salicylate, magnesium salicylate and calcium salicylate, quaternary ammonium salts, benzalkonium chloride, benzethonium chloride, trimethyltetradecylammonium bromide (cetrimide), and cyclodextrins and their adducts. One or more charge-control agents may be used. Charge-control agent may be present, for example, in an amount of up to 10% by weight, especially at least 1% by weight, for example from 1-2% by weight, based on the total weight of the powder material.

The powder material may also include a flow aid present at the outer surface of the powder particles to reduce the cohesive and/or other forces between the particles. Suitable flow aids (which are also known as “surface additives”) are, for example, colloidal silica; metal oxides, e.g. fumed titanium dioxide, zinc oxide or alumina; metal stearates, e.g. zinc, magnesium or calcium stearate; talc; functional and non-functional waxes; and polymer beads, e.g. polymethyl methacrylate beads, fluoropolymer beads and the like. Such materials may also enhance tribocharging. A mixture of flow aids, for example silica and titanium dioxide, should especially be mentioned. The powder material may contain, for example, 0 to 3% by weight, advantageously at least 0.1%, e.g. 0.2 to 2.5%, by weight of surface additive flow aid.

The powder material may also include a dispersing agent, for example a lecithin. The dispersing component is preferably a surfactant which may be anionic, cationic or non-ionic, but may be another compound which would not usually be referred to as a “surfactant” but has a similar effect. The dispersing component may be a co-solvent. The dispersing component may be one or more of, for example, sodium lauryl sulphate, docusate sodium, Tweens (sorbitan fatty acid esters), polyoxamers and cetostearyl alcohol. Preferably, the powder material includes at lest 0.5%, e.g. at least 1%, for example from 2% to 5%, by weight of dispersing component, based on the weight of the powder material.

Preferably, the powder material has a glass transition temperature (Tg) in the range of 40° C. to 180° C., e.g. in the range 40 to 120° C. Advantageously; the material has a Tg in the range of 50° C. to 100° C. A preferred minimum Tg is 55° C., and a preferred maximum Tg is 70° C. Accordingly, more advantageously, the material has a Tg in the range of 55° C. to 70° C.

Preferably, at least 50% by volume of the particles of the material have a particle size no more than 100 μm. Advantageously, at least 50% by volume of the particles of the material have a particle size in the range of 5 μm to 40 μm. More advantageously, at least 50% by volume of the particles of the material have a particle size in the range of 10 to 25 μm.

Powder having a narrow range of particle size should especially be mentioned. Particle size distribution may be quoted, for example, in terms of the Geometric Standard Deviation (“GSD”) figures d₉₀/d₅₀ or d₅₀/d₁₀ where d₉₀ denotes the particle size at which 90% by volume of the particles are below this figure (and 10% are above), d₁₀ represents the particle size at which 10% by volume of the particles are below this figure (and 90% are above), and d₅₀ represents the mean particle size. Advantageously, the mean (d₅₀) is in the range of from 5 to 40 μm, for example from 10 to 25 μm. Preferably, d₉₀/d₅₀ is no more than 1.5, especially no more than 1.35, more especially no more than 1.32, for example in the range of from 1.2 to 1.5, especially 1.25 to 1.35, more especially 1.27 to 1.32, the particle sizes being measured, for example, by Coulter Counter. Thus, for example, the powder may have d₅₀=10 μm, d₉₀−13 μm d₁₀=7 μm, so that d₉₀/d₅₀=1.3 and d₅₀/d₁₀=1.4.

The invention will now be described in further detail by way of example only by reference to the accompanying drawings in which

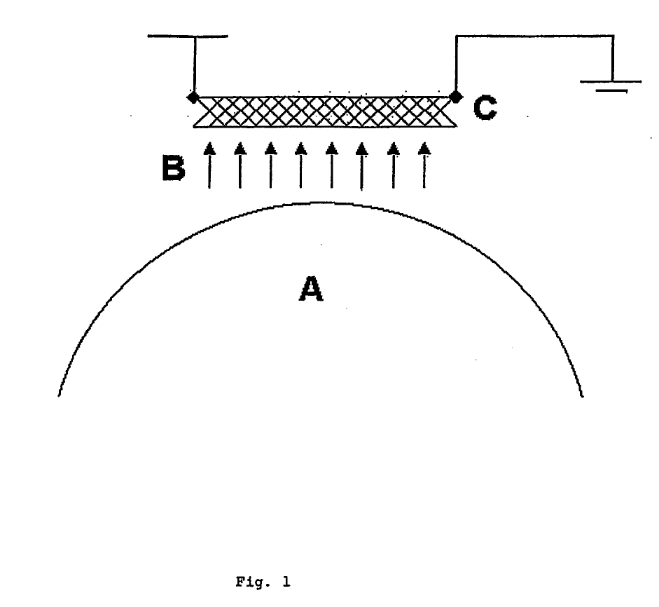

FIG. 1 shows a schematic view of a part of an apparatus suitable for carrying out the process of the invention.

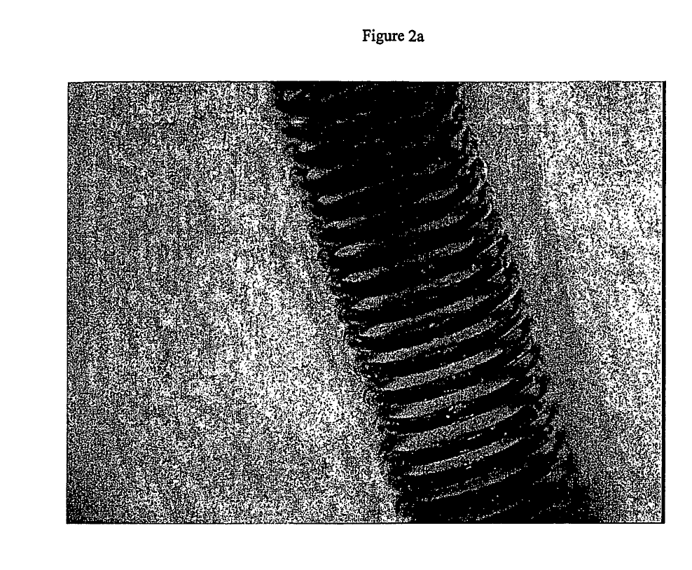

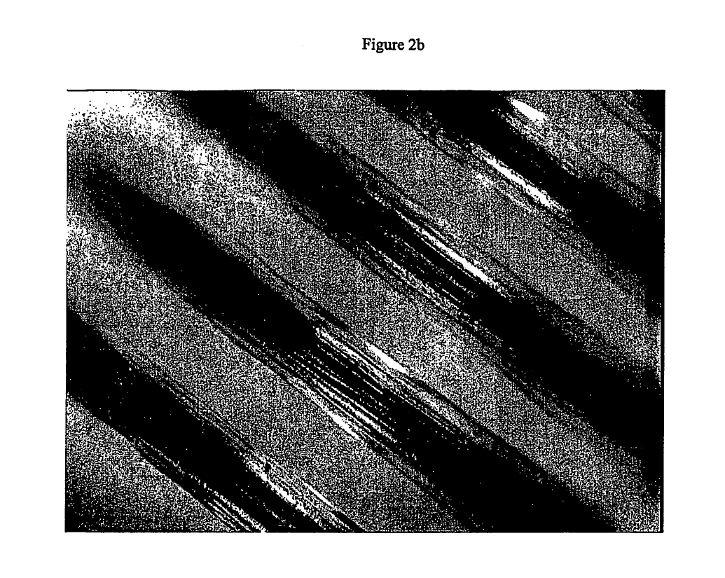

FIGS. 2a and 2b show images (magnified) of a copper coil coated in accordance with the electrostatic powder deposition process of the invention.

In FIG. 1 a powder delivery system A incorporating a source of charged powder is provided adjacent to but spaced apart from a stent C. A voltage source is connected to apply in this particular example a positive voltage to the powder delivery system whilst the support for the stent is maintained at earth potential. As previously described, the potentials applied may comprise both DC bias potentials and an AC potential. The powder is also charged to a positive potential. In use powder is caused to move across from the powder source of the powder delivery system to the stent C as a result of the interaction of the charged powder with the electric field. The powder transferring across is illustrated by the arrows B in FIG. 1. The stent C is rotated by means not shown to ensure coating on all sides of the stent. As charged powder is transferred to the stent C, so the electric field between the powder delivery system and the stent is reduced. If desired, the application of the positive voltage can be maintained with the stent rotating until the electric field is reduced to such a low level that powder ceases to transfer across from the powder source.

After application the powder deposited on the stent is heated by an IR heater (not shown) to convert the powder into a continuous layer, and is then allowed to cool to provide a coated stent.

FIGS. 2a and 2b show, as an example, magnified images of a copper coil, approximately 3 mm in diameter coated with a powder comprising poly (butyl methacrylate-co-methyl methacrylate) and having a particle size 100% less than 53 μm. The spacing of the closest part of the coil to the powder source was 1 mm. The coil was coated using a 3000V DC field for 60 seconds and the coated coil was fused under a hot air stream with a set temperature of 200° C. for 30 seconds. The coating thickness was approximately 50 microns. In this instance, the fuser temperature was chosen to achieve a fast fusion. Fusion of the material could be achieved at lower temperatures, for example approximately 120° C. for 90 seconds. A shorter coating time could also be achieved by rotating the coil.

1. A method for the coating of a surgical device, wherein the coating is carried out by electrostatic powder deposition.

2. A method as claimed in claim 1, wherein after application the powder is heated to form a coherent coating layer.

3. A method for coating a device for implantation in the human or animal body or for a medical interventional procedure, wherein the coating is carried out by electrostatic powder deposition and subsequently the powder is heated to form a coherent coating layer.

4. A method as claimed in claim 1, wherein the powder material comprises a polylactide, polycaprolactone, polyvinylpyrrolidone, poly(acrylic acid), polyurethane or poly(butyl methacrylate-co-methyl methacrylate).

5. A method as claimed in claim 1, wherein the powder material is applied from a source spaced from the device by a distance in the range of 0.5 mm to 5 mm.

6. A method as claimed in claim 1, including the steps of
applying a bias voltage to generate an electric field between a source of the powder material and the device;
applying the electrostatically charged powder material to the device, the powder material being driven onto the device by the interaction of the electric field with the charged powder material and the presence of the charged powder material on the device serving to build up an electric charge on the device and thereby reduce the electric field generated by the bias voltage between the source of powder material and the device, and continuing the application of the electrostatically charged powder material to the device until the electric field between the source of powder material and the device is so small that the driving of the powder material by the electric field onto the substrate is substantially terminated.

7. A method as claimed in claim 1, wherein the device is for delivery of an active material and that active material is contained in the coating.

8. A method as claimed in claim 1, wherein the device is for delivery of a diagnostic agent and that diagnostic agent is contained in the coating.

9. A method as claimed in claim 1, wherein the coating includes a source of radioactivity.

10. A method as claimed in claim 1, wherein the coating includes an agent for the treatment or prevention of restenosis, or an anticoagulant, an anti-thrombogenic agent, an anti-microbial agent, an anti-neoplastic agent, an antiplatelet agent, an immunosuppressant agent, an antimetabolite, an anti-proliferative agent, or an anti-inflammatory agent.

11. A method as claimed in claim 1, wherein the device is a stent.

12. A method as claimed in claim 1, wherein the device is a heart valve.

13. A method as claimed in claim 1, wherein the device is a pacemaker, catheter, orthopaedic or dental implant, artificial hip or other joint, artificial organ, neurostimulator, cardiovert defibrillator, dialysis tubing or tubing for heart-lung machine.

14. A method as claimed in claim 1, wherein the device is made of metal.

15. A device as specified in claim 1, which has been coated by a method as claimed in claim 1.

16. A method as claimed in claim 1, which comprises application of a DC bias potential and an AC potential.

17. A method as claimed in claim 16, wherein the AC potential is substantially higher than the DC potential.

18. A method as claimed in claim 16, wherein the powder material is applied from a source spaced from the device by a distance in the range of 0.5 mm to 5 mm.

19. A method as claimed in claim 17, wherein the powder material is applied from a source spaced from the device by a distance in the range of 0.5 mm to 5 mm.

20. A method as claimed in claim 6, wherein the powder material is applied from a source spaced from the device by a distance in the range of 0.5 mm to 5 mm.

21. A method as claimed in claim 6, wherein the bias voltage is a DC voltage and an AC voltage is also applied.

22. A method as claimed in claim 21, wherein the alternating voltage has a peak to peak value greater than the peak value of the DC bias voltage.

23. A method as claimed in claim 22, wherein the alternating voltage has a peak to peak value more than twice the peak value of the DC bias voltage.

24. A method as claimed in claim 21, wherein the powder material is applied from a source spaced from the device by a distance in the range of 0.5 mm to 5 mm.

25. A method as claimed in claim 22, wherein the powder material is applied from a source spaced from the device by a distance in the range of 0.5 mm to 5 mm.

26. A method as claimed in claim 23, wherein the powder material is applied from a source spaced from the device by a distance in the range of 0.5 mm to 5 mm.
